(19) United States
(12) Patent Application Publication
(10) Pub. No.: US 2025/0177621 A1
(43) Pub. Date: Jun. 5, 2025

(54) SENSOR ASSEMBLY, EXTRACORPOREAL CIRCULATION MANAGEMENT DEVICE, AND EXTRACORPOREAL CIRCULATION MANAGEMENT METHOD

(71) Applicant: TERUMO KABUSHIKI KAISHA, Tokyo (JP)

(72) Inventors: Takashi Saito, Fujinomiya-shi (JP); Junji Inoue, Fujinomiya-shi (JP)

(21) Appl. No.: 19/051,512

(22) Filed: Feb. 12, 2025

Related U.S. Application Data
(63) Continuation of application No. PCT/JP2023/032331, filed on Sep. 5, 2023.

(30) Foreign Application Priority Data
Sep. 7, 2022 (JP) JP20220141903

(51) Int. Cl. A61M 1/14; A61M 1/16; G01L 27/00

(52) U.S. Cl. CPC A61M 1/154; A61M 1/1698; G01L 27/005; A61M 2205/3344; A61M 2205/702

(57) ABSTRACT

A sensor assembly stores calibration information thereon even after a blood circuit is filled with fluid. A pressure sensor element 42 measures a value of pressure in a fluid flow channel 45. A storage unit 51 which is integrated with sensor element 42 stores, as calibration information, a calibration value determined in advance according to a value indicated by the pressure sensor element 42 in a state in which the pressure sensor element 42 is opened to atmospheric pressure and in which the internal pressure in a fluid flow channel 45 of the pressure sensor element 42 is equal to the atmospheric pressure. Measured pressure values and the calibration value are provided to a control unit which adjusts the measured values on the basis of the calibration information.

### CROSS REFERENCE TO RELATED APPLICATIONS

This application is a continuation of PCT Application No. PCT/JP2023/032331, filed Sep. 5, 2023, based on and claiming priority to Japanese Application No. JP20220141903, filed Sep. 7, 2022, both of which are incorporated herein by reference in their entirety.

### BACKGROUND OF THE INVENTION

The present invention relates to a sensor assembly, an extracorporeal circulation management device, and an extracorporeal circulation management method.

In the related art, percutaneous cardiopulmonary support (PCPS) has been provided for cardiopulmonary resuscitation, circulatory support, and respiratory support in emergency treatment. The percutaneous cardiopulmonary support is a method of temporarily assisting and substituting a cardiopulmonary function using extracorporeal membrane oxygenation (ECMO).

An extracorporeal circulation management device such as extracorporeal membrane oxygenation includes a blood circuit provided with, for example, a centrifugal pump, an oxygenator, a blood removal channel, and a blood supply channel and exchanges gases in blood removed and supplies the blood to the blood supply channel.

In such an extracorporeal circulation management device, it is recommended to measure the pressure of the blood circuit for management and safety of a patient. As a pressure gauge for measuring pressure, a gauge pressure sensor that measures a difference from atmospheric pressure is generally used (see, for example, JP2014-119324A).

In a pressure sensor such as a gauge pressure sensor, it is necessary to open the pressure sensor to atmospheric pressure before use and calibrate (zero-value correction) a deviation unique to the pressure sensor due to a variation in resistance of the strain gauge. Therefore, the gauge pressure sensor needs to branch off from the blood circuit and be disposed at a point where both sides of the gauge pressure sensor can be opened to the atmospheric pressure.

On the other hand, from the viewpoint of using the extracorporeal circulation management device for a long time, the pressure sensor is preferably built in the blood circuit because a branched branch pipe may lead to thrombus formation.

However, in a configuration in which the pressure sensor is built in the blood circuit, once priming is performed and the blood circuit is filled with fluid, a differential pressure is applied, and thus calibration (zero-value correction) cannot be performed correctly thereafter.

### SUMMARY OF THE INVENTION

The present invention has been made to solve the above problem, and an object of the present invention is to provide a sensor assembly, a extracorporeal circulation management device, and a extracorporeal circulation management method capable of performing calibration even after a blood circuit is filled with fluid.

The above-described object of the present invention is achieved by the following means.

(1) A sensor assembly is provided including a pressure sensor element that is capable of measuring a value of pressure to be applied; and a storage unit that stores, as calibration information, a value indicated by the pressure sensor element in a state where the pressure sensor element is opened to atmospheric pressure and the internal pressure in a fluid flow channel of the pressure sensor element is equal to the atmospheric pressure.

(2) The sensor assembly according to (1) may further include a plurality of the pressure sensor elements; and an integration unit that integrates cables connected to the plurality of pressure sensor elements, in which the storage unit is provided in the integration unit.

(3) The sensor assembly according to (1) or (2), may further include a transmission unit that is capable of transmitting the calibration information stored in the storage unit to the outside.

(4) The sensor assembly according to (3) may include the transmission unit as a wireless transmission unit.

(5) The invention may further comprise an extracorporeal circulation management device including the sensor assembly according to any one of (1) to (4); and a calibration unit that acquires the calibration information stored in the storage unit, and continuously calibrates a value actually measured by the pressure sensor element on the basis of the calibration information that has been acquired.

(6) The invention may further comprise an extracorporeal circulation management method including a step of setting, as calibration information, a value indicated by a pressure sensor element in a state where the pressure sensor element is opened to atmospheric pressure and the internal pressure in a fluid flow channel of the pressure sensor element is equal to the atmospheric pressure, and a stop of continuously calibrating a value actually measured by the pressure sensor element on the basis of the calibration information.

According to the sensor assembly and the extracorporeal circulation management device configured as described above, the storage unit stores, as calibration information, a value indicated by the pressure sensor element in a state where the pressure sensor element is opened to atmospheric pressure and the internal pressure in the fluid flow channel of the pressure sensor element is equal to the atmospheric pressure. Therefore, even after a blood circuit is filled with fluid, the value actually measured by the pressure sensor element can be continuously calibrated on the basis of the calibration information, and calibration can be performed.

In addition, the extracorporeal circulation management method described above includes the step of setting, as calibration information, a value indicated by the pressure sensor element in a state where the pressure sensor element is opened to atmospheric pressure and the internal pressure in the fluid flow channel of the pressure sensor element is equal to the atmospheric pressure, and continuously calibrating a value actually measured by the pressure sensor element on the basis of the calibration information. Therefore, even after the blood circuit is filled with fluid, the value actually measured by the pressure sensor element can be continuously calibrated on the basis of the calibration information, and calibration can be performed.

### BRIEF DESCRIPTION OF THE DRAWINGS

FIG. 1 is a system diagram illustrating an extracorporeal circulation management device according to a first embodiment of the present invention.

FIG. 2 is a schematic view illustrating a sensor assembly according to the first embodiment.

FIG. 3 is a perspective view illustrating a sensor unit of the sensor assembly according to the first embodiment.

FIG. 4 is a front view of the sensor unit illustrated in FIG. 3.

FIG. 5 is a cross-sectional view taken along line 5-5 of FIG. 4.

FIG. 6 is a front cross-sectional view of the sensor unit illustrated in FIG. 3.

FIG. 7A and FIG. 7B are graphs illustrating a state of calibration by a calibration unit.

FIG. 8 is a system diagram illustrating an extracorporeal circulation management device according to a second embodiment of the present invention.

FIG. 9 is a view of a sensor assembly according to the second embodiment, the view corresponding to FIG. 5.

FIG. 10 is a system diagram illustrating an extracorporeal circulation management device according to a third embodiment of the present invention.

### DETAILED DESCRIPTION OF PREFERRED EMBODIMENTS

Hereinafter, embodiments of the present invention will be described with reference to the accompanying drawings. Note that the following description does not limit the technical scope or the significance of each term disclosed in the claims. Furthermore, dimensional ratios of the drawings are exaggerated for illustration purpose and may differ from actual ratios.

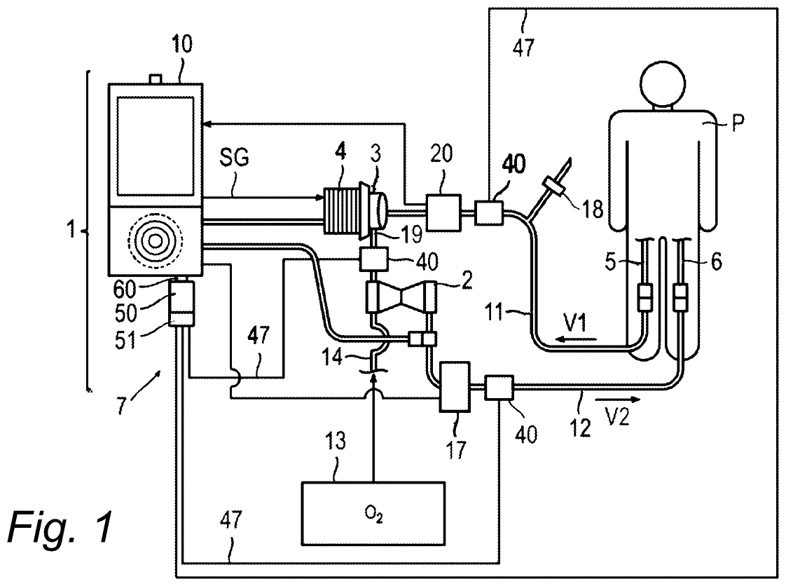

FIG. 1 is a system diagram illustrating an extracorporeal circulation management device 1 used for percutaneous cardiopulmonary support (PCPS) in order to temporarily assist and substitute cardiopulmonary function of a patient with a weakened heart until recovery of heart function.

According to the extracorporeal circulation management device 1, it is possible to perform surgery using a Veno-Arterial (VA) system. In this surgery, a pump is actuated to remove blood from a vein (vena cava) of a patient, and an oxygenator 2 exchanges gases in the blood to oxygenate the blood, thereby returning the blood to an artery (aorta) of the patient. This extracorporeal circulation management device 1 is used for assisting the heart and lungs. Hereinafter, a surgery to remove blood from a patient and perform predetermined treatment outside the body and return the blood into the patient's body will be referred to as “extracorporeal circulation”.

As illustrated in FIG. 1, the extracorporeal circulation management device 1 includes a blood circuit for circulating blood. The blood circuit includes the oxygenator 2, a centrifugal pump 3, a drive motor 4 as a drive means that drives the centrifugal pump 3, a venous catheter (percutaneous catheter for blood removal) 5, an arterial catheter (catheter for blood supply) 6, a sensor assembly 7 that measures the pressure of blood flowing through the blood circuit, and a controller 10 as a control unit.

The venous catheter (catheter for blood removal) 5 is inserted from the femoral vein, and through the inferior vena cava, a distal end of the venous catheter 5 is retained in the right atrium. The venous catheter 5 is connected to the centrifugal pump 3 through a blood removal tube (blood removal line) 11. The blood removal tube 11 is a channel for supplying blood.

The arterial catheter (catheter for blood supply) 6 is inserted from the femoral artery.

The drive motor 4 actuates the centrifugal pump 3 according to a command SG from the controller 10, causing the centrifugal pump 3 to remove blood from a patient P through the blood removal tube 11 and supply the blood to the oxygenator 2, and then, return the blood to the patient P through a blood supply tube (blood supply line) 12.

The oxygenator 2 is disposed between the centrifugal pump 3 and the blood supply tube 12. The oxygenator 2 exchanges gases in blood (oxygenates and/or decarbonates blood). The oxygenator 2 is, for example, a membrane oxygenator, and particularly preferably, a hollow fiber membrane oxygenator. To this oxygenator 2, an oxygen gas is supplied from an oxygen gas supply unit 13 through a tube 14. The blood supply tube 12 is a channel that connects the oxygenator 2 and the arterial catheter 6.

The blood removal tube 11 and the blood supply tube 12 may employ a channel including, for example, a synthetic resin such as vinyl chloride resin and silicone rubber having high transparency and flexibility that enables elastic deformation. In the blood removal tube 11, blood, or a fluid, flows in V1 direction. In the blood supply tube 12, blood flows in V2 direction.

In the blood circuit illustrated in FIG. 1, an ultrasonic bubble detection sensor 20 is disposed in a part of the blood removal tube 11. A fast clamp 17 is disposed in a part of the blood supply tube 12.

The tube 11 (12 and 19) of the blood circuit in the extracorporeal circulation management device 1 is provided with the sensor assembly 7. As described later, the sensor assembly 7 includes three sensor units 40. The sensor units 40 can be attached to, for example, a fixing point in the blood removal tube 11, a fixing point in the blood supply tube 12 of the blood circuit, and a fixing point in a connecting tube 19 that connects the centrifugal pump 3 and the oxygenator 2. Accordingly, it is possible to measure pressure inside the tube 11 (12 and 19) by the sensor assembly 7 during the extracorporeal circulation performed on the patient P by the extracorporeal circulation management device 1. Note that the fixing point of the sensor assembly 7 is not limited to the aforementioned fixing points and the sensor assembly 7 may be attached to any points in the blood circuit.

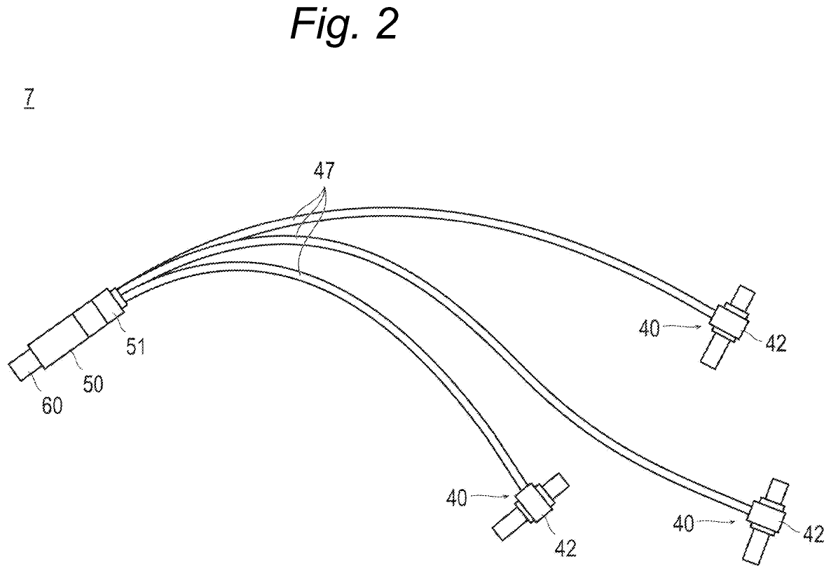

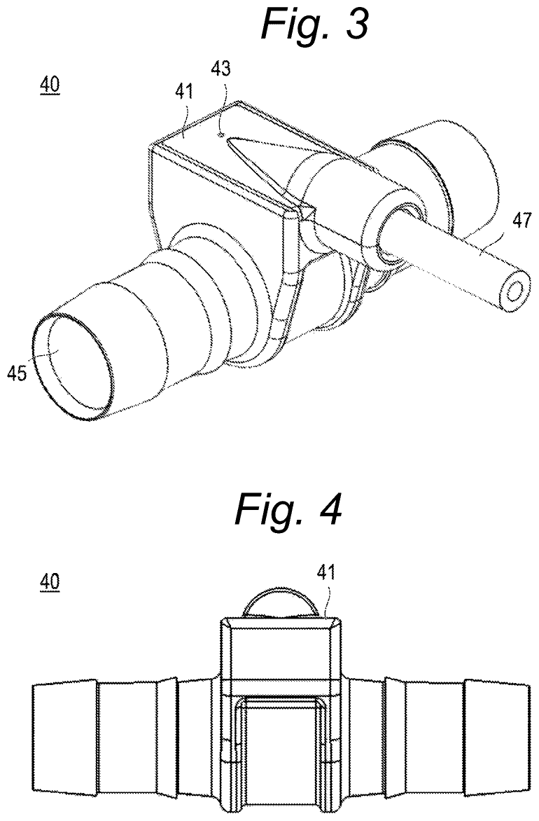

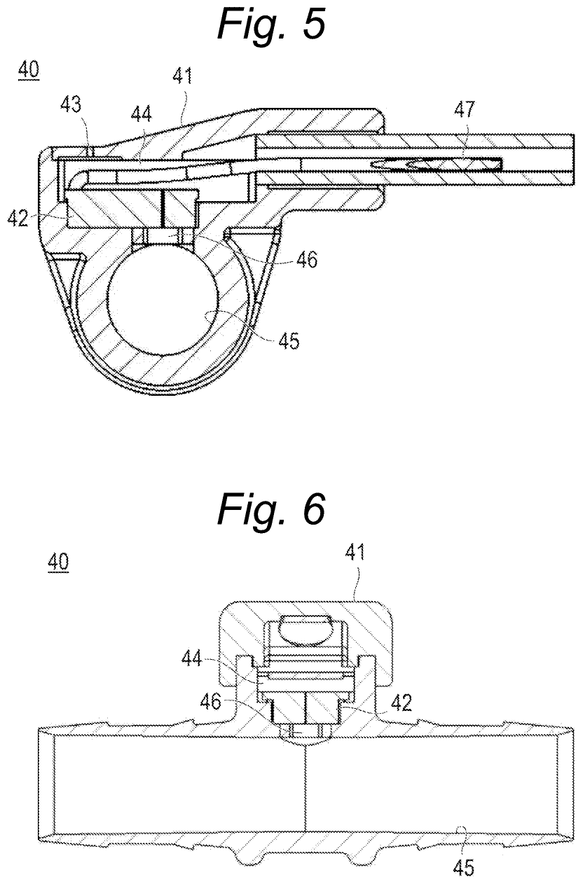

Hereinafter, the configuration of the sensor assembly 7 will be described in detail with reference to FIGS. 2 to 6. FIG. 2 is a schematic view illustrating the sensor assembly 7 according to the first embodiment. FIG. 3 is a perspective view illustrating the sensor unit 40 of the sensor assembly 7 according to the first embodiment. FIG. 4 is a front view of the sensor unit 40 illustrated in FIG. 3. FIG. 5 is a cross-sectional view taken along line 5-5 of FIG. 4. FIG. 6 is a front cross-sectional view of the sensor unit 40 illustrated in FIG. 3.

As illustrated in FIG. 2, the sensor assembly 7 includes the three sensor units 40, cables 47 connected to the sensor units 40, an integration unit 50 that integrates the cables 47, and a transmission unit 60.

The sensor unit 40 is a so-called pressure sensor. As illustrated in FIG. 1, the sensor unit 40 functions as a connector connected to the blood circuit. The sensor unit 40 measures the pressure of the fluid filled in the sensor unit 40.

As illustrated in FIGS. 3 to 6, the sensor unit 40 includes a main body 41, a pressure sensor element 42 disposed inside the main body 41, a hole 43 formed in the main body 41, an atmospheric pressure space 44 defined by the main body 41 and the pressure sensor element 42, a fluid flow channel 45 formed at a lower part of the main body 41, and a gel 46 disposed under the pressure sensor element 42 and transmitting the pressure of the fluid flowing through the fluid flow channel 45 to the pressure sensor element 42.

As illustrated in FIG. 5, the main body 41 includes the atmospheric pressure space 44 and the fluid flow channel 45. The main body 41 is configured as a connector connected to the blood circuit of the extracorporeal circulation management device 1.

The pressure sensor element 42 can measure the value of pressure applied to the pressure sensor element 42. The pressure sensor element 42 includes a strain gauge. The pressure sensor element 42 is connected to the transmission unit 60 via the cable 47, and the value of pressure measured by the pressure sensor element 42 is transmitted to the controller 10 via the transmission unit 60.

As illustrated in FIG. 5, the hole 43 is formed in an upper part of the main body 41. The atmospheric pressure space 44 of the main body 41 and outside air communicate with each other through the hole 43.

In the sensor unit 40 configured as described above, the pressure of the fluid flowing through the blood circuit is measured by the pressure sensor element 42 via the gel 46 when the fluid flows through the fluid flow channel 45 as a relative pressure with respect to the atmospheric pressure. Specifically, the pressure of the fluid flowing through the blood circuit is converted into an electrical signal by the pressure sensor element 42 and transmitted to the controller 10 via the cable 47 and the transmission unit 60.

As illustrated in FIG. 2, the integration unit 50 integrates the three cables 47 connected to the three pressure sensor elements 42. As illustrated in FIG. 2, a storage unit 51 is mounted on the integration unit 50. The storage unit 51 is a so-called memory chip.

The storage unit 51 stores, as calibration information, a value indicated by the pressure sensor element 42 in a state where the pressure sensor element 42 is opened to atmospheric pressure and the internal pressure in the fluid flow channel 45 of the pressure sensor element 42 is equal to the atmospheric pressure. Here, the “state where the pressure sensor element 42 is opened to atmospheric pressure and the internal pressure in the fluid flow channel 45 of the pressure sensor element 42 is equal to the atmospheric pressure” is specifically at the time of shipment of a product.

The transmission unit 60 is provided on a side of the integration unit 50 opposite to the side on which the cables 47 are provided. As illustrated in FIG. 1, the transmission unit 60 is mechanically and electrically connected to the controller 10. The transmission unit 60 transmits the indicated value as the calibration information stored in the storage unit 51 of the integration unit 50 to the controller 10.

The controller 10 includes an acquisition unit that acquires the calibration information stored in the storage unit 51, and a calibration unit that continuously calibrates the value actually measured by the pressure sensor element 42 on the basis of the calibration information acquired by the acquisition unit.

Next, an extracorporeal circulation management method by the extracorporeal circulation management device 1 according to the first embodiment will be described.

First, at the time of shipment, the sensor assembly 7 in which the value indicated by the pressure sensor element 42 is stored as calibration information in the storage unit 51 is prepared.

Next, the sensor unit 40 of the sensor assembly 7 is connected to a portion of the blood circuit of the extracorporeal circulation management device 1 where the pressure is measured. Then, priming is performed in the blood circuit.

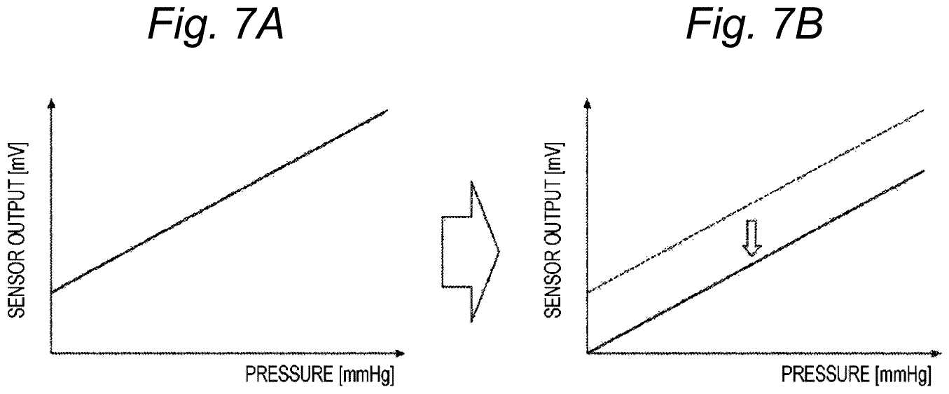

Next, while blood is circulated in the extracorporeal circulation management device 1, the sensor assembly 7 measures the pressure of the blood passing through the sensor unit 40 of the sensor assembly 7. At this time, as illustrated in FIG. 7(A), output of the sensor unit 40 has a deviation unique to the pressure sensor due to the variation in resistance of the strain gauge.

At this time, the acquisition unit of the controller 10 receives the indicated value as the calibration information stored in the storage unit 51, and the calibration unit of the controller 10 continuously calibrates (i.e., adjusts) the value actually measured by the pressure sensor element 42 as illustrated in FIG. 7(B) (see the arrow).

Here, for example, in the case of a configuration in which the indicated value cannot be stored as the calibration information, a differential pressure is applied to the pressure sensor element 42 from start to finish unless it is before the fluid is filled, that is, before priming is performed, and thus, the timing of zero calibration with respect to the atmospheric pressure is lost. In contrast, the extracorporeal circulation management device 1 according to the present embodiment stores, as calibration information, the indicated value at the time of being open to the atmospheric pressure under a controlled environment such as a manufacturer's shipping process and is shipped, and the controller 10 recalls the value and appropriately calculates the value, so that the automatically calibrated value can be displayed. Therefore, calibrated measurement can be performed even after the blood circuit is filled with the fluid.

As described above, the sensor assembly 7 according to the present embodiment includes: the pressure sensor element 42 that is capable of measuring a value of pressure to be applied; and the storage unit 51 that stores, as calibration information, a value indicated by the pressure sensor element 42 in a state where the pressure sensor element 42 is opened to atmospheric pressure and the internal pressure in the fluid flow channel 45 of the pressure sensor element 42 is equal to the atmospheric pressure. According to the sensor assembly 7 configured as described above, the storage unit 51 stores, as calibration information, a value indicated by the pressure sensor element 42 in a state where the pressure sensor element 42 is opened to atmospheric pressure and the internal pressure in the fluid flow channel 45 of the pressure sensor element 42 is equal to the atmospheric pressure. Therefore, even after the blood circuit is filled with fluid, the value actually measured by the pressure sensor element 42 can be continuously calibrated on the basis of the calibration information, and calibration can be performed on the measured value.

In addition, the three pressure sensor elements 42 are provided, the sensor assembly 7 further includes the integration unit 50 that integrates the cables 47 connected to the three pressure sensor elements 42, and the storage unit 51 is provided in the integration unit 50. According to the sensor assembly 7 configured as described above, since the cables 47 are integrated by the integration unit 50, it is possible to put the cables 47 together, and it is possible to suppress complication in the extracorporeal circulation management device 1. In addition, it is possible to save labor for the operator to perform preparation work of connecting the cable 47 to each of the three pressure sensor elements 42 to be connected to the extracorporeal circulation management device 1. Furthermore, prevention of an erroneous connection of the three cables 47 can be expected.

In addition, the sensor assembly 7 further includes the transmission unit 60 that transmits the calibration information stored in the storage unit 51 to the controller 10. According to the sensor assembly 7 configured as described above, calibration of measured values can be more suitably performed even after the blood circuit is filled with the fluid.

In addition, as described above, the extracorporeal circulation management device 1 according to the present embodiment includes: the sensor assembly 7 described above; and the calibration unit that acquires the calibration information stored in the storage unit 51, and continuously calibrates the value actually measured by the pressure sensor element 42 on the basis of the calibration information that has been acquired. According to the extracorporeal circulation management device 1 configured as described above, calibration of measured values can be performed even after the blood circuit is filled with the fluid.

In addition, as described above, the extracorporeal circulation management method according to the present embodiment includes: a step of setting, as calibration information, a value indicated by the pressure sensor element 42 in a state where the pressure sensor element 42 is opened to atmospheric pressure and the internal pressure in the fluid flow channel 45 of the pressure sensor element 42 is equal to the atmospheric pressure, and continuously calibrating the value actually measured by the pressure sensor element 42 on the basis of the calibration information. According to the extracorporeal circulation management method, even after the blood circuit is filled with the fluid, the value actually measured by the pressure sensor element 42 can be continuously calibrated on the basis of the calibration information, and calibration can be performed.

### Second Embodiment

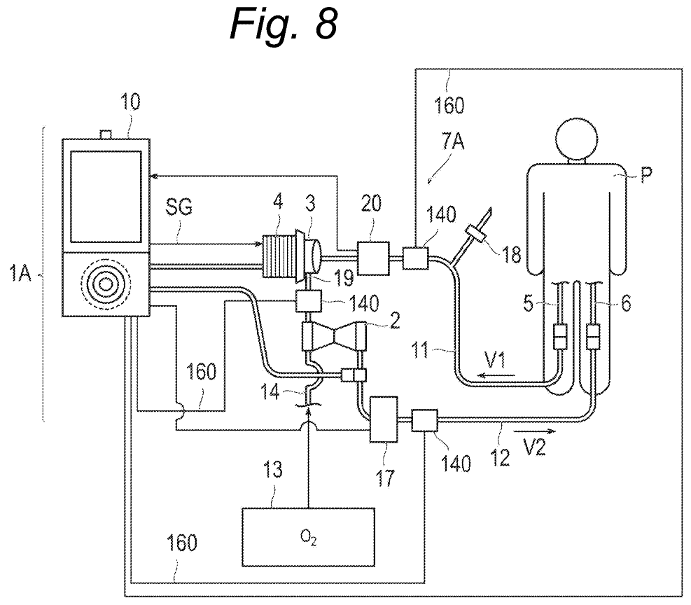

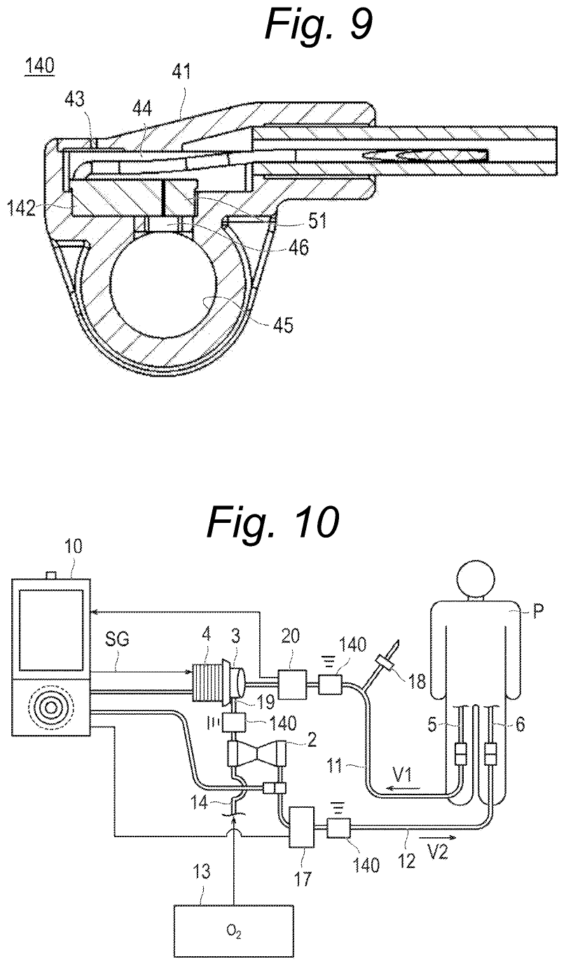

Next, an extracorporeal circulation management device 1A according to a second embodiment of the present invention will be described with reference to FIGS. 8 and 9.

FIG. 8 is a system diagram illustrating the extracorporeal circulation management device 1A according to the second embodiment of the present invention. FIG. 9 is a view of a sensor assembly 7A according to the second embodiment, the view corresponding to FIG. 5.

Configurations common to the first embodiment will not be described, and configurations characteristic of the second embodiment will be described. Note that the same members as those of the first embodiment will be denoted by the same reference numerals, and redundant description will be omitted. The second embodiment is different from the first embodiment in a location where a storage unit 51 is provided, and the like.

As illustrated in FIG. 8, the sensor assembly 7A according to the second embodiment includes three sensor units 140, and three transmission units 160 connected to the three sensor units 140. The sensor assembly 7A according to the second embodiment is not provided with an integration unit.

The transmission unit 160 is a transmission cable. As illustrated in FIG. 8, the sensor unit 140 is electrically connected to a controller 10 via the transmission unit 160 that is a transmission cable.

As illustrated in FIG. 9, the sensor unit 140 includes a main body 41, a pressure sensor element 142 disposed inside the main body 41, a hole 43 formed in the main body 41, an atmospheric pressure space 44 defined by the main body 41 and the pressure sensor element 142, a fluid flow channel 45 formed at a lower part of the main body 41, and a gel 46 disposed under the pressure sensor element 42 and transmitting the pressure of the fluid flowing through the fluid flow channel 45 to the pressure sensor element 42.

In the sensor assembly 7A according to the second embodiment, the pressure sensor element 142 also functions as the storage unit 51.

In the sensor unit 140 configured as described above, the pressure of the fluid flowing through the blood circuit is measured by the pressure sensor element 42 via the gel 46 when the fluid flows through the fluid flow channel 45 as a relative pressure with respect to the atmospheric pressure. Specifically, the pressure of the fluid flowing through the blood circuit is converted into an electrical signal by the pressure sensor element 42 and transmitted to the controller 10 via the transmission unit 160. Further, the indicated value as calibration information stored in the storage unit 51 is transmitted to the controller 10 via the transmission unit 160.

Since the extracorporeal circulation management method of the extracorporeal circulation management device 1A according to the second embodiment is the same as the extracorporeal circulation management method of the extracorporeal circulation management device 1 according to the first embodiment, the description thereof will be omitted.

Although the sensor assembly according to the present invention has been described through the embodiments, the present invention is not limited to the configurations described in the embodiments and modifications thereof and is appropriately changed based on the claims.

For example, in the second embodiment described above, the transmission unit 160 is a wired transmission cable, but as illustrated in FIG. 10, a transmission unit may be wireless communication. A method of the wireless communication is not particularly limited, but for example, a radio frequency identifier (RFID) can be used.

In addition, in the first embodiment and the second embodiment described above, the three pressure sensor elements 42, 142 are provided, but the number of pressure sensor elements is not limited to three.

In addition, in the first embodiment and the second embodiment described above, the sensor units 40, 140 are connected to the blood circuit, but a sensor unit may be mounted in the oxygenator 2.

### REFERENCE SIGNS LIST

1, 1A Extracorporeal circulation management device
2 Oxygenator
7, 7A Sensor assembly
10 Controller
40, 140 Sensor unit
42, 142 Pressure sensor element
45 Fluid flow channel
50 Integration unit
51 Storage unit
60, 160 Transmission unit

What is claimed is:

1. A sensor assembly for an extracorporeal circulation device, comprising:
a pressure sensor element configured to measure a value of pressure in a fluid flow channel; and
a storage unit that stores, as calibration information, a calibration value indicated by the pressure sensor element in a calibration state in which the pressure sensor element is opened to an atmospheric pressure and in which an internal pressure in the fluid flow channel of the pressure sensor element is equal to the atmospheric pressure;
wherein the sensor assembly is adapted to communicate with a control unit of the extracorporeal circulation device in order to provide the calibration value and the measured value of pressure.

2. The sensor assembly according to claim 1, further comprising:
a plurality of the pressure sensor elements; and
an integration unit that integrates cables connected to the plurality of pressure sensor elements;
wherein the storage unit is provided in the integration unit, and wherein the storage unit stores a plurality of calibration values for the respective pressure sensor elements.

3. The sensor assembly according to claim 1, further comprising a transmission unit that transmits the calibration information stored in the storage unit to the control unit.

4. The sensor assembly according to claim 3, wherein the transmission unit is comprised of a wireless transmission unit.

5. An extracorporeal circulation management device comprising:
a sensor assembly which comprises:
a pressure sensor element configured to measure a value of pressure in a fluid flow channel; and
a storage unit that stores, as calibration information, a calibration value indicated by the pressure sensor element in a calibration state in which the pressure sensor element is opened to an atmospheric pressure and in which an internal pressure in the fluid flow channel of the pressure sensor element is equal to the atmospheric pressure; and
a control unit that acquires the measured value of pressure from the pressure sensor element and acquires the calibration information stored in the storage unit, wherein the control unit continuously adjusts a pressure value actually measured by the pressure sensor element on a basis of the calibration information that has been acquired.

6. The extracorporeal circulation management device according to claim 5, further comprising:
a plurality of the pressure sensor elements; and
an integration unit that integrates cables connected to the plurality of pressure sensor elements;
wherein the storage unit is provided in the integration unit, and wherein the storage unit stores a plurality of calibration values for the respective pressure sensor elements.

7. The extracorporeal circulation management device according to claim 5, further comprising a transmission unit that transmits the calibration information stored in the storage unit to the control unit.

8. The extracorporeal circulation management device according to claim 7, wherein the transmission unit is comprised of a wireless transmission unit.

9. A method of managing an extracorporeal circulation device, the method comprising the steps of:
setting a calibration value, as calibration information, which is indicated by a pressure sensor element in a calibration state in which the pressure sensor element is opened to an atmospheric pressure and in which an internal pressure in a fluid flow channel of the pressure sensor element is equal to the atmospheric pressure;
storing the calibration value in a storage unit integrated with the pressure sensor element as a sensor assembly;
transmitting the calibration value and the measured value of pressure to a control unit of the extracorporeal circulation device; and
the control unit continuously adjusting the pressure value actually measured by the pressure sensor element on a basis of the calibration information.

10. The method of claim 9:
wherein the extracorporeal circulation device includes a plurality of the pressure sensor elements and an integration unit which integrates cables connected to the plurality of pressure sensor elements; and
wherein the storage unit is provided in the integration unit, and wherein the storage unit stores a plurality of calibration values for the respective pressure sensor elements.
